(19) United States
(12) Patent Application Publication
(10) Pub. No.: US 2020/0163868 A1
(43) Pub. Date: May 28, 2020

(54) Oral Care Compositions Comprising Charcoal

(71) Applicant: Colgate-Palmolive Company, New York, NY (US)

(72) Inventors: Melissa MARTINETTI, Bridgewater, NJ (US); David SURIANO, Edison, NJ (US); Yun XU, Langhorne, PA (US); Gayatri PATEL, Edison, NJ (US)

(73) Assignee: Colgate-Palmolive Company, New York, NY (US)

(21) Appl. No.: 16/683,663

(22) Filed: Nov. 14, 2019

Related U.S. Application Data
(60) Provisional application No. 62/772,198, filed on Nov. 28, 2018.

(51) Int. Cl. A61K 8/97; A61K 8/25; A61Q 11/00

(52) U.S. Cl. CPC A61K 8/97; A61Q 11/00; A61K 8/25

(57) ABSTRACT

Disclosed herein is an oral care composition comprising at least one abrasive and a charcoal agent, wherein the charcoal agent is present in an amount ranging from about 0.02% to about 4% by weight based on the total weight of the oral care composition. Further disclosed herein are methods of making an oral care composition comprising at least one abrasive and at least one charcoal agent wherein the charcoal agent is present in an amount ranging from about 0.02% to about 4% by weight based on the total weight of the oral care composition, as well as methods of whitening teeth and/or cleaning the teeth comprising contacting the teeth with an oral care composition as disclosed herein.

### BACKGROUND

A tooth is comprised of an inner dentin layer and an outer hard enamel layer that is the protective layer of the tooth. The enamel layer of a tooth is naturally an opaque white or slightly off-white color, and is composed of hydroxyapatite mineral crystals that create a somewhat porous surface. It is this enamel layer that can become stained or discolored, and it is believed that the porous nature of the enamel layer is what allows staining agents and discoloring substances to permeate the enamel and discolor the tooth.

Many substances that a person confronts or comes in contact with on a daily basis can stain or reduce the whiteness of their teeth. In particular, the pigments produced by oral microbes and pigments found in foodstuffs, beverages such as coffee, tea and soda, tobacco products, and medicaments tend to stain the teeth. These products or substances may accumulate on the enamel layer of the tooth and form a pellicle film over the teeth. The staining and discoloring substances can then permeate the porous enamel layer. This problem occurs gradually over many years, but imparts a noticeable discoloration of the enamel of one's teeth. Discolored teeth are widely considered to be cosmetically unattractive. There is therefore significant consumer demand for oral care compositions with tooth whitening properties.

A number of methods for whitening teeth are known. Of these, the use of tooth-whitening toothpastes is perhaps the most common. Most tooth-whitening toothpastes can be administered by consumers without the intervention of a dental professional. Although many whitening toothpastes may comprise a chemical for whitening, such as a peroxide, many whitening toothpastes also or alternatively comprise an abrasive, which polishes the teeth when applied by brushing. The polishing action serves to remove stains from the surface of the teeth.

The abrasive, however, should be carefully selected such that it produces a whitening effect without damaging the tooth by excessively abrading the enamel or dentin. Certain common abrasives, such as calcium carbonate, have relatively low abrasiveness. While they may contribute to the cleaning of teeth, they fail to produce an appreciable whitening effect due to their low abrasiveness. In contrast, other abrasives may be relatively strong depending on their concentration in the formulation, and may be overly abrasive, resulting in damage to the enamel.

As consumers may be cautious about damage to teeth resulting from tooth whitening products, it is desirable to know the relative abrasivity of various toothpaste formulations. One measurement for such abrasivity is the Relative Dentin Abrasion (RDA) of toothpaste formulation. The American Dental Association (ADA) considers dentifrices having an RDA of less than 250 safe and effective. Stain removal efficacy may be measured, in turn, by the Pellicle Cleaning Ratio (PCR). In general, dentifrices having a high PCR and a low RDA are desirable. For whitening toothpastes that rely on physical removal of extrinsic stains, teeth whitening efficacy strongly correlates to the toothpaste abrasiveness (i.e., high RDA) in most cases. Thus, it is therefore desirable to reduce toothpaste abrasiveness while still maintaining a high stain removal efficacy.

### BRIEF SUMMARY

Disclosed herein is an oral care composition comprising at least one abrasive and a charcoal agent, wherein the charcoal agent is present in an amount of about 4% or less, such as an amount ranging from about 0.02% to about 4% or from about 0.5% to about 3% by weight based on the total weight of the oral care composition. Although charcoal is known to have abrasive properties, it is surprisingly and unexpectedly disclosed herein that, in certain oral care compositions, the addition of charcoal to a composition comprising at least one abrasive may actually lower the abrasivity of the compound while simultaneously maintaining or increasing the composition's stain removal and whitening efficacy.

In certain embodiments, the at least one abrasive is a silica abrasive, such as high cleaning silica abrasives and standard cleaning silica abrasives. In certain embodiments, the at least one abrasive is present in the oral care composition in an amount ranging from about 5% to about 25%, by weight relative to the total weight of the oral care composition, and in certain embodiments disclosed herein, the charcoal agent is present in the oral care composition in an amount ranging from about 0.5% to about 3%, by weight relative to the total weight of the oral care composition.

According to various embodiments of the disclosure, the oral care composition disclosed herein has a PCR greater than about 90, such as a PCR greater than about 100. In various other embodiments, the oral care composition disclosed herein has an RDA of less than about 200, such as less than about 150, less than about 100, or less than about 50. In certain embodiments, the oral care composition disclosed herein has a ratio of PCR to RDA ranging from about 0.5 to about 2.5, such as from about 1.0 to about 2.5.

Further disclosed herein a method of making an oral care composition comprising mixing at least one orally acceptable vehicle, at least one abrasive, and a charcoal agent to form an oral care composition, wherein the charcoal agent is present in an amount ranging from about 0.02% to about 4% by weight based on the total weight of the oral care composition.

In certain embodiments of the methods disclosed herein, the at least one abrasive is a silica abrasive, such as high cleaning silica abrasives and standard cleaning silica abrasives. In certain embodiments of the methods disclosed herein, the at least one abrasive is present in the oral care composition in an amount ranging from about 5% to about 25%, by weight relative to the total weight of the oral care composition, and in certain embodiments disclosed herein, the charcoal agent is present in the oral care composition in an amount ranging from about 0.5% to about 3%, by weight relative to the total weight of the oral care composition.

In various embodiments of the methods disclosed herein, the oral care composition has a PCR of greater than about 90, such as a PCR greater than about 100, and in various embodiments, the oral care composition has an RDA of less than about 200, such as less than about 150, less than about 100, or less than about 50. In certain embodiments of the methods disclosed herein, the oral care composition has a ratio of PCR to RDA ranging from about 0.5 to about 2.5, such as from about 1.0 to about 2.5.

Further disclosed herein is a method of whitening teeth and/or a method of cleaning teeth, comprising contacting the teeth with an oral care composition comprising at least one abrasive and a charcoal agent, wherein the charcoal agent is present in an amount ranging from about 0.02% to about 4% by weight based on the total weight of the oral care composition. In various embodiments, the step of contacting the teeth comprises brushing the oral care composition against the teeth.

Further areas of applicability of the present disclosure will become apparent from the detailed description provided hereinafter. It should be understood that the detailed description and specific examples, while indicating certain preferred embodiments of the disclosure, are intended for purposes of illustration only and are not intended to limit the scope of the disclosure.

### DETAILED DESCRIPTION

The following description of the preferred embodiments is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

As used herein, the term “one or more of” with respect to a listing of items such as, for example, A and B, means A alone, B alone, or A and B. The term “at least one of” is used to mean one or more of the listed items can be selected.

Unless otherwise specified, all percentages and amounts expressed herein and elsewhere in the specification should be understood to refer to percentages by weight. The amounts given are based on the active weight of the material.

Disclosed herein is an oral care composition comprising a charcoal agent and at least one abrasive. The oral care composition disclosed herein may be in the form of any known dentifrice, including for example, in the form of a toothpaste or a gel.

### At Least One Abrasive

The oral care compositions disclosed herein comprise at least one abrasive. Useful abrasives may include, for example, silicas, aluminum oxide, aluminum silicate, calcined alumina, bentonite or other siliceous materials, insoluble phosphates, perlite, pumice, calcium carbonate, polymer particulates, and mixtures thereof. Additional exemplary abrasives may include, for example, calcium phosphate abrasives, such as tricalcium phosphate (Ca₃(PO₄)₂), hydroxyapatite (Ca₁₀(PO₄).6(OH)₂), dicalcium phosphate dihydrate (CaHPO₄.2H₂O, also sometimes referred to herein as DiCal), and calcium pyrophosphate; sodium metaphosphate; and potassium metaphosphate.

In certain embodiments, the at least one abrasive is a silica. Abrasive silica is a relatively strong abrasive that is known to produce a tooth whitening effect. In certain embodiments, the abrasive silica is optionally a precipitated or hydrated silica having a mean particle size of up to about 20 microns, such as Zeodent® 103, 105, 113, 114, 115, 119, or 124 marketed by J.M. Huber Chemicals Division, Havre de Grace, Md. 21078, or Sylodent® 783 marketed by Davison Chemical Division of W.R. Grace & Company. Other possible abrasive silicas include silica gels and precipitated amorphous silica, such as those having an oil adsorption value of less than 100 cc/100 g and optionally in the range of from about 45 cc/100 g to less than about 70 cc/100 g silica. These silicas are colloidal particles having an average particle size ranging from about 3 microns to about 12 microns, such as between about 5 microns to about 10 microns or between about 3 microns to about 4 microns.

Abrasive silicas are distinct from thickening silicas. In general, abrasive (cleaning) silicas can be characterized as having oil absorption levels of about 40 to 150 cc/100 g and having an Einlehner abrasion of 3 or greater mg loss/100,000 revolutions, whereas thickening abrasives have oil absorption levels of greater than 150 cc/100 g and have an Einlehner abrasion of less than 2 mg loss/100,000 revolutions.

Thickening silicas can include silicas such as Zeodent® 163 (oil absorption levels of 190 cc/100 g and having an Einlehner abrasion of less than 2 mg loss/100,000 revolutions), Zeodent® 165 (oil absorption levels of 220 cc/100 g and having an Einlehner abrasion of less than 2 mg loss/100,000 revolutions), Zeodent® 167 (oil absorption levels of 235 cc/100 g and having an Einlehner abrasion of less than 2 mg loss/100,000 revolutions) marketed by J.M. Huber Chemicals Division, Havre de Grace, Md. 21078.

The amount of the at least one abrasive may be selected so as to avoid excessive abrasion of the tooth surface. The abrasiveness of a composition may be measured using, for example, the radioactive dentine abrasion (RDA) test method or other methods known in the art.

RDA is a relative measure of abrasivity. Typically, extracted human or cow teeth are irradiated in a neutron flux, mounted in methylmethacrylate (bone glue), stripped of enamel, inserted into a brushing-machine, brushed by American Dental Association (ADA) standards, which include a reference toothbrush, 150 grams of pressure, 1500 strokes, and a 4-to-1 water-toothpaste slurry. The radioactivity of the rinse water is then measured and recorded. For experimental control, the test is repeated with an ADA reference toothpaste made of calcium pyrophosphate, with this measurement given a value of 100 to calibrate the relative scale. In certain embodiments, the RDA of the oral care composition disclosed herein may be less than about 250, such as less than about 200, less than about 150, less than about 125, less than about 100, less than about 75, or less than about 50. In certain embodiments, the RDA of the oral care composition disclosed herein may range from less than about 250 to about 20, such as from less than about 200 to about 20, less than about 150 to about 20, less than about 125 to about 20, less than about 100 to about 20, less than about 75 to about 20, less than about 50 to about 20, or less than about 250 to about 50,

The abrasives disclosed herein, including silica abrasives, generally have an average particle size ranging about 0.1 micron to about 30 microns, such as about 5 microns to about 15 microns. The silica abrasives can be from precipitated silica or silica gels, such as the silica xerogels described in U.S. Pat. No. 3,538,230, to Pader et al. and U.S. Pat. No. 3,862,307, to Digiulio, both incorporated herein by reference. Particular silica xerogels are marketed under the trade name Syloid® by the W. R. Grace & Co., Davison Chemical Division, for example.

The particle size of the at least one abrasive may be measured using any means known in the art, including, for example, using a Malvern Particle Size Analyzer, Model Mastersizer S. This instrument, manufactured by Malvern Instruments, Inc., Southborough, Mass., United States of America, as disclosed in U.S. Pat. No. 6,290,933, the contents of which are incorporated herein by reference.

Suitable abrasive silica for use in the compositions disclosed herein may have an Einlehner hardness ranging from about 3 to about 20 mg loss/100,000 revolutions, such as from about 5 to about 15 mg loss/100,000 revolutions, and an oil absorption value of about 40 to less than about 150 cc/100 g. The Einlehner hardness may be determined using an Einlehner At-1000 Abrader. Using this method, a Fourdrinier brass wire screen is weighed and exposed to the action of a 10% aqueous silica suspension for a given number of revolutions. The hardness value is expressed as milligrams of weight lost of the Fourdrinier wire screen per 100,000 revolutions.

The oil absorption value may be determined using ASTM rub-out method D281. Low oil absorption silica abrasives that may be mentioned include those marketed under the trade designation Sylodent® XWA by Davison Chemical Division of W.R. Grace & Co., Baltimore, Md. 21203. Sylodent® 650 XWA, a silica hydrogel composed of particles of colloidal silica having a water content of 29% by weight averaging from 7 to 10 microns in diameter, and an oil absorption of less than 70 cc/100 g of silica is also an example of a low oil absorption silica abrasive that may be used in the oral care compositions disclosed herein.

Silicas suitable for use in the composition may also have a BET surface area of about 100 to 700 m²/g of silica. The BET surface area may be determined by a BET nitrogen adsorption method described in Brunauer et al., Journal of the American Chemical Society, 60, 309 (1938), the contents of which are incorporated herein by reference. The BET measurement may be performed using an Accelerated Surface Area and Porosimetry Analyzer (ASAP 2400), by Micromeritics Instrument Corporation, Norcross, Ga., United States of America. The sample is outgassed under vacuum at 350° C. for a minimum of two hours before measurement.

Any suitable amount of abrasive, such as silica abrasive, can be employed. In certain embodiments, the oral care compositions disclosed herein will comprise an effective amount of the at least one abrasive so as to provide a stain removal, tooth whitening and/or cleaning effect. In certain embodiments, the at least one abrasive is present in the composition in an amount ranging from about 10% to about 45%, such as from about 18% to about 40%, from about 20% to about 30%, about 15%, about 20%, about 25%, about 30%, about 35%, or about 40%, by weight relative to the total weight of the composition. In certain exemplary embodiments, the total amount of the at least one abrasive present in the composition is about 20% by weight of the composition, and in certain embodiments, the total amount of the silica abrasive present in the composition ranges from about 10% to about 45%, such as from about 18% to about 40% or from about 20% to about 30%.

### Charcoal Agent

In addition to at least one abrasive agent, the oral care compositions disclosed herein further comprise a charcoal agent. The charcoal agent can be any known charcoal composition. In certain embodiments, the charcoal agent may be a charcoal powder, such as vegetable carbon. Charcoal is a fine, black powder that may be made from various materials, including, for example, vegetable materials, coconut shells, bone char, olive pits, coal, and sawdust. Charcoal, which has a porous texture, may serve to trap various substances, such as toxins and acidic materials. The charcoal agent may be present in the oral care compositions disclosed herein in any amount effective for tooth whitening. As charcoal is known to be abrasive, however, it should not be incorporated into the oral care compositions disclosed herein in amounts that would result in a high RDA, such as an RDA over about 250. In certain embodiments the charcoal agent may be present in the oral care composition in an amount of about 10% or less, such as about 4% or less, or such as an amount ranging from about 0.02% to about 10%, such as from about 0.2% to about 4%, about 0.5% to about 3%, about 0.1%, about 0.2%, about 0.3%, about 0.4%, about 0.5%, about 1%, about 2%, or about 3%, by weight relative to the total weight of the oral care composition.

As charcoal is a known abrasive, one of ordinary skill in the art would anticipate that adding charcoal to a composition comprising at least one additional abrasive agent would enhance the abrasivity of the composition, for example by increasing the RDA of the composition. As disclosed herein, however, it has been surprisingly and unexpectedly discovered that the addition of a charcoal agent, such as a charcoal agent present in the composition in an amount ranging from about 0.02% to about 4%, not only increases or maintains the cleaning efficacy of the composition (as measured, for example, by PCR), but may also serve to reduce the abrasivity of the composition, e.g., by reducing the RDA. There is thus observed a synergistic effect between the charcoal agent and the at least one abrasive, such as the at least one silica abrasive, wherein the abrasivity from the abrasive is lowered when the amount of charcoal is increased. While not wishing to be bound by theory, it is postulated that the particles of the charcoal agent may interact with the at least one abrasive to reduce the abrasive's rolling resistance over a tooth's enamel. This may result in lower friction and less abrasivity between the abrasive and the enamel, while not decreasing the composition's ability to remove pellicle and stain, thus maintaining or enhancing the whitening efficacy of the composition.

The RDA value is determined according to the method recommended by the American Dental Association as set forth by Hefferren, Journal of Dental Research, 55:4, 1976, 563-573, and described U.S. Pat. Nos. 4,340,583; 4,420,312; and 4,421,527, the contents of each of which are incorporated herein by reference. In summary, an irradiated dentin surface is treated with the slurried composition to be evaluated and the level of radioactivity present in the slurry post treatment is indicative of the level of wear to the dentin surface.

As used herein, pellicle cleaning ratio (PCR) is a measure of the effectiveness of the dentifrice to remove stains. In certain embodiments, the oral care compositions disclosed herein may have a PCR ranging from about 75 to about 110, such as from about 85 to about 105. PCR values may be determined as measured by the method described U.S. Pat. Nos. 5,658,553 and 5,651,958, the contents of which are incorporated herein by reference. In summary, a clear pellicle material is applied to a bovine tooth which is then stained with a combination of the pellicle material and tea, coffee, and FeCl₃, which is subsequently treated with the composition, and the change in the reflectance of the tooth surface before and after treatment is the PCR value.

As disclosed herein, the addition of a charcoal agent, such as about 0.02% to about 4% by weight of a charcoal agent, to the compositions disclosed herein comprising at least one abrasive, results in an oral care composition that can provide enhanced cleaning and/or whitening benefits coupled with low abrasion of the tooth enamel. The result is a relatively high ratio of PCR to RDA. In certain embodiments, the oral care compositions disclosed herein have a ratio of PCR to RDA that is greater than 0.5, such as greater than about 0.6, greater than about 1.0, greater than about 1.5, greater than about 2.0, or greater than about 2.5. In certain embodiments, the oral care compositions disclosed herein have a ratio of PCR to RDA that ranges from about 0.5 to about 2.5, such as about 0.6 to about 2.3, about 0.8 to about 2.0, or about 1.0 to about 1.8. In certain embodiments, the addition of charcoal to a composition comprising at least one abrasive, such as a silica abrasive, lowers the RDA of the resultant oral care composition compared to the composition without charcoal, and in certain embodiments, the addition of charcoal to the composition comprising at least one abrasive raises or maintains the PCR compared to the composition without charcoal. In various exemplary embodiments, the addition of charcoal to a composition comprising at least one abrasive, such as a silica abrasive, raises the ratio of PCR to RDA of the resultant oral care composition compared to the composition without charcoal.

### Additional Ingredients

The compositions of the present invention may comprise one or more additional oral care ingredients. The one or more additional oral care ingredients may optionally be selected from the group consisting of: surfactants, desensitizing agents, whitening agents, tartar control agents, binders, thickeners, detergents, adhesion agents, foam modulators, pH modifying agents, mouth feel agents, sweeteners, flavorants, colorants, preservatives, humectants, fluoride sources and combinations thereof.

Surfactants may be used in the oral care compositions of the present invention to provide foaming, taste, flavor, texture and mouth feel properties to the compositions, and in particular to render the compositions more cosmetically acceptable. Suitable surfactants include without limitation water-soluble salts of C₈₋₂₀ alkyl sulfates, sulfonated monoglycerides of C₈₋₂₀ fatty acids, sarcosinates, taurates, sodium lauryl sulfate, lauryl glucoside, sodium cocoyl monoglyceride sulfonate, sodium cocoyl glutamate, sodium lauryl sarcosinate, sodium lauryl isoethionate, sodium laureth carboxylate and sodium dodecyl benzenesulfonate, and cocoamidopropyl betaine. In certain embodiments, the surfactant comprises sodium lauryl sulfate (SLS).

The compositions disclosed herein optionally incorporate at least one desensitizing agent, such as potassium salts (including potassium nitrate, potassium bicarbonate, potassium chloride, potassium citrate, and potassium oxalate); capsaicin; eugenol; strontium salts; zinc salts; chloride salts and combinations thereof. Such desensitizing agents may be added in any effective amounts, such as an amount ranging from about 1% to about 20% by weight based on the total weight of the composition, depending on the desensitizing agent chosen. The compositions disclosed herein may also be used to treat hypersensitivity by blocking dentin tubules when applied to a tooth.

The compositions disclosed herein may optionally include a tooth whitening or tooth bleaching agent in addition to the at least one abrasive and the charcoal agent. Suitable whitening and bleaching agents include, for example, peroxides, metal chlorites, and persulfates. Peroxides include hydroperoxides, hydrogen peroxide, peroxides of alkali and alkaline earth metals, organic peroxy compounds, peroxy acids, and mixtures thereof. Peroxides of alkali and alkaline earth metals include lithium peroxide, potassium peroxide, sodium peroxide, magnesium peroxide, calcium peroxide, barium peroxide, and mixtures thereof. Other peroxides include perborate, urea peroxide, and mixtures thereof. Suitable metal chlorites may include calcium chlorite, barium chlorite, magnesium chlorite, lithium chlorite, sodium chlorite, and potassium chlorite. Such tooth whitening or tooth bleaching agents may be added in any effective amount, such as from about 1% to about 20% by weight based on the total weight of the composition.

The oral care compositions disclosed herein may optionally include tartar control agents such as pyrophosphate salts including dialkali or tetraalkali metal pyrophosphate salts such as tetrasodium pyrophosphate (Na₄P₂O₇), K₄P₂O₇, Na₂K₂P₂O₇, Na₂H₂P₂O₇ and K₂H₂P₂O₇ sodium tripolyphosphate, long chain polyphosphates such as sodium hexametaphosphate, and cyclic phosphates such as sodium trimetaphosphate.

The compositions disclosed herein may further comprise a binder. Any conventional binder may be utilized. Suitable agents include marine colloids; carboxyvinyl polymers; carrageenans; starches; cellulosic polymers such as hydroxyethylcellulose, carboxymethylcellulose (cannellose), hydroxypropyl methyl cellulose, and salts thereof (e.g., cannellose sodium); natural gums such as karaya, xanthan, gum arabic and tragacanth; chitosan; colloidal magnesium aluminum silicate; and colloidal silica. In certain embodiments, a binder is present in the composition in an amount ranging from about 0.5% to about 5% by weight of the composition.

Thickening agents suitable for use in the compositions disclosed herein may include natural and synthetic gums and colloids. Suitable thickening agents include naturally occurring polymers such as carrageenan, xanthan gum, polyglycols of varying molecular weights sold under the tradename Polyox®, and polyvinylpyrrolidone. Compatible inorganic thickening agents include amorphous silica compounds that may function as thickening agents and include colloidal silicas compounds available under the trade designation Cab-o-sil® manufactured by Cabot Corporation and distributed by Lenape Chemical, Bound Brook, N.J., Zeodent 165 from J. M. Huber Chemicals Division, Havre de Grace, Md. 21078; and Sylodent® 15, available from Davison Chemical Division of W. R. Grace Corporation, Baltimore, Md. 21203. Other inorganic thickening agents include natural and synthetic clays such as hectorite clays, lithium magnesium silicate (laponite), and magnesium aluminum silicate (such as Veegum®).

The oral care compositions disclosed herein may optionally comprise at least one adhesion agent. The adhesion agent may by a polymeric adherent material. The polymeric adherent material attaches to the surface of a mammalian tooth and/or to the heterogeneous biofilm which also may be present on a tooth's surface. Attachment may occur by any means, such as ionic interaction, van der Waals forces, and hydrophobic-hydrophilic interactions. The adherent material may be, for example, any homopolymers or copolymers (hereinafter referred to collectively as a “polymers”) that adhere to the surface of a tooth. Such polymers may include cellulose polymers, for example one or more hydroxyalkyl cellulose polymers, such as hydroxypropylmethyl cellulose (HPMC), hydroxyethylpropyl cellulose (HEPC), hydroxybutylmethyl cellulose (HBMC), carboxymethyl cellulose (CMC). In certain embodiments, the polymeric adherent material comprises at least one cellulose material, for example sodium carboxymethyl cellulose.

The polymeric adherent material may alternatively or additionally include poly (ethylene oxide) polymers (such as Polyox® from Dow Chemical), linear PVP and cross-linked PVP, PEG/PPG copolymers (such as BASF Pluracare® L1220), ethylene oxide (EO)-propylene oxide (PO) block copolymers (such as polymers sold under the name Pluronic® available from BASF Corporation), ester gum, shellac, pressure sensitive silicone adhesives (such as BioPSA® from Dow-Corning), methacrylates, or mixtures thereof. In an embodiment, a copolymer comprises (PVM/MA). Optionally, the copolymer may be selected from poly (methylvinylether/maleic anhydride), poly (methylvinylether/maleic acid), poly (methylvinylether/maleic acid) half esters, and poly (methylvinylether/maleic acid) mixed salts.

The oral care compositions disclosed herein also may include at least one foam modulator. Foam modulators may function to increase the amount of foam produced, for example, when the oral cavity is brushed using an oral care composition as disclosed herein. Illustrative examples of foam modulators that increase the amount of foam include, but are not limited to, polyoxyethylene and certain polymers such as alginate polymers. The polyoxyethylene may increase the amount of foam and the thickness of the foam generated by the oral care carrier component included in the oral care composition. Polyoxyethylene is also known as polyethylene glycol (“PEG”) or polyethylene oxide. The polyoxyethylenes suitable for use in the compositions disclosed herein may have a molecular weight ranging from about 200,000 to about 7,000,000, such as from about 600,000 to about 2,000,000 or from about 800,000 to about 1,000,000. Polyox® is the trade name for the high molecular weight polyoxyethylene produced by Union Carbide.

The polyoxyethylene may be present in the compositions disclosed herein in an amount ranging from about 1% to about 90%, such as from about 5% to about 50% or from about 10% to about 20%, by weight based on the total weight of the oral care carrier component of the oral care composition. In certain embodiments, the at least one foaming agent may be present in the oral care composition in an amount ranging from about 0.01% to about 0.9%, such as from about 0.05% to about 0.5%, or from about 0.1% to about 0.2% by weight based on the total weight of the composition.

The compositions disclosed herein may comprise at least one pH modifying agent. Such pH modifying agents may include acidifying agents to lower pH, basifying agents to raise pH, and buffering agents to control pH within a desired range. For example, one or more compounds selected from acidifying, basifying and buffering agents can be included to provide a pH ranging from about 2 to about 10, or in various illustrative embodiments, a pH ranging from about 2 to about 8, about 3 to about 9, about 4 to about 8, about 5 to about 7, about 6 to about 10, or about 7 to about 9. Any orally acceptable pH modifying agent can be used including, without limitation, carboxylic, phosphoric and sulfonic acids, acid salts (such as monosodium citrate, disodium citrate, and monosodium malate); alkali metal hydroxides such as sodium hydroxide; carbonates such as sodium carbonate, bicarbonates, and sesquicarbonates; borates; silicates; phosphates (such as monosodium phosphate, trisodium phosphate, and pyrophosphate salts), imidazole and the like. The at least one pH modifying agent may be present in the oral care composition in a total amount effective to maintain the composition in an orally acceptable pH range.

Mouth-feel agents that may be used herein include materials which impart a desirable texture or other feeling during use of the composition. Such mouth-feel agents may include bicarbonate salts, which may impart a “clean feel” to teeth and gums due to effervescence and release of carbon dioxide. Any orally acceptable bicarbonate can be used, including without limitation alkali metal bicarbonates such as sodium and potassium bicarbonates, ammonium bicarbonate, and mixtures thereof. Mouth-feel agents such as bicarbonate salts may be present in the oral care composition in an amount ranging from about 0.1% to about 50%, such as from about 1% to about 20% by weight based on the total weight of the oral care composition.

The compositions disclosed herein may further comprise at least one sweetener. Sweeteners which may be used include, for example, artificial sweeteners such as saccharin, acesulfam, neotam, cyclamate, and sucralose; natural high-intensity sweeteners such as thaumatin, stevioside, and glycyrrhizin; and sugar alcohols such as sorbitol, xylitol, maltitol, and mannitol. The at least one sweetener may be present in the oral care composition in an amount ranging from about 0% to about 0.2%, such as from about 0.005% to about 0.1%, by weight based on the total weight of the composition.

The compositions of the present invention may optionally comprise at least one flavorant. Flavorants that may be used in the compositions disclosed herein include essential oils as well as various flavoring aldehydes, esters, alcohols, and similar materials. Examples of the essential oils include oils of spearmint, peppermint, aniseed, wintergreen, sassafras, clove, sage, eucalyptus, marjoram, cinnamon, lemon, lime, grapefruit, and orange. Flavorants may also include such chemicals as menthol, carvone, and anethole. The flavorant may be incorporated in the compositions disclosed herein in an amount ranging from about 0.1% to about 5%, such as from about 0.5% to 1.5%, by weight based on the total weight of the composition.

The compositions disclosed herein may further comprise at least one colorant. Colorants may include pigments, dyes, lakes, and agents imparting a particular luster or reflectivity such as pearling agents. Any orally-acceptable colorant can be used, including without limitation talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride, and the like. The at least one colorant may be present in the composition in an amount ranging from about 0.001% to about 20%, such as from about 0.01% to about 10%, or from about 0.1% to about 5%, by weight based on the total weight of the composition.

Preservatives, such as chlorhexidine, triclosan, quaternary ammonium compounds (such as benzalkonium chloride) or parabens (such as methyl or propyl paraben) may also be incorporated in the compositions disclosed herein. The amount of preservative may, for example, range from about 0% to about 0.5%, such as from about 0.05% to about 0.1% by weight based on the total weight of the composition.

In certain embodiments, the oral care compositions disclosed herein further comprise at least one fluoride ion source. Fluoride ion sources include, but are not limited to: stannous fluoride, sodium fluoride, potassium fluoride, potassium monofluorophosphate, sodium monofluorophosphate, ammonium monofluorophosphate, sodium fluorosilicate, ammonium fluorosilicate, amine fluoride such as olaflur (N′-octadecyltrimethylendiamine-N,N,N′-tris(2-ethanol)-dihydrofluoride), ammonium fluoride, and combinations thereof. Optionally, the fluoride ion source includes stannous fluoride, sodium fluoride, amine fluorides, sodium monofluorophosphate, as well as mixtures thereof. In certain embodiments, the oral care compositions disclosed herein may also contain a source of fluoride ions or fluorine-providing ingredient in amounts sufficient to supply about 50 to about 5000 ppm fluoride ion, such as from about 100 to about 1000, from about 200 to about 500, or about 250 ppm fluoride ion. Fluoride ion sources may be added to the compositions disclosed herein at a level in ranging from about 0.001% to about 10%, such as from about 0.003% to about 5%, 0.01% to about 1%, or about 0.05%, by weight based on the total weight of the composition. However, it is to be understood that the weights of fluoride salts to provide the appropriate level of fluoride ion may vary based on the weight of the counter ion in the salt, and one of skill in the art may readily determine such amounts. In certain embodiments, the oral care compositions disclosed herein are substantially free of a fluoride ion source, such as substantially free of sodium monofluorophosphate. As used herein, “substantially free of” indicates that the composition does not contain the ingredient or contains no effective amount of the ingredient.

### Methods of Making Oral Care Compositions

Disclosed herein are methods of making an oral care composition comprising mixing at least one orally acceptable vehicle, at least one abrasive, and a charcoal agent to form an oral care composition, wherein the charcoal agent is present in an amount ranging from about 0.02% to about 4% by weight based on the total weight of the oral care composition. In certain embodiments, the oral care composition is a gel, and in certain embodiments, the oral care composition is a paste.

As used herein, the term “orally acceptable vehicle” refers to a material that is safe for use in the composition with which other ingredients may be associated without affecting their efficacy. Such orally acceptable carriers may be selected for compatibility with the other ingredients of the oral care composition. Selection of specific orally acceptable carrier components may dependent on the desired product form, including pastes, rinses, gels, and paints. In certain embodiments, the at least one orally acceptable carrier comprises water, and in certain embodiments, the at least one orally acceptable carrier is anhydrous. Exemplary orally acceptable carriers may include water, sorbitol, glycerin, and mixtures thereof. Other exemplary orally acceptable carriers may include polymers and/or copolymers of polyethylene glycol, ethylene oxide propylene oxide, silicone, and mixtures thereof.

### Methods of Teeth Whitening

Further disclosed herein is a method for whitening teeth of a subject comprising contacting the teeth with an oral care composition comprising at least one abrasive and a charcoal agent, wherein the charcoal agent is present in an amount ranging from about 0.02% to about 4% by weight based on the total weight of the oral care composition. In certain embodiments, contacting the teeth comprises brushing the oral care composition against the teeth, for example with an implement such as a toothbrush. In other embodiments, disclosed herein are methods of cleaning the teeth of a subject or methods of polishing the teeth of a subject, wherein the methods comprise the teeth with an oral care composition comprising at least one abrasive and a charcoal agent, wherein the charcoal agent is present in an amount ranging from about 0.02% to about 4% by weight based on the total weight of the oral care composition.

In certain embodiments, the subject is a human, or a companion animal such as a cat, a dog or a horse. The composition may be applied by the any suitable method known in the art. The composition may be applied to the oral cavity of the subject using any suitable technique known in the art. The technique may vary depending on the nature of the composition. For example, the composition may be applied by brushing, such as by brushing for about 2 minutes.

Any appropriate dosage regime may be used in combination with the methods disclosed herein. For example, the composition may be applied to the oral cavity of the subject once a day, twice a day, or more often. In certain embodiments, the composition is applied to the oral cavity of the subject twice a day. The subject may be treated with the composition for a period of at least one day, at least one month, at least six months, at least one year, or for a lifetime.

In addition to teeth whitening, various diseases and disorders of the oral cavity may be treated or prevented using the methods and compositions disclosed herein. Optionally, the methods and compositions disclosed herein may be used to treat or prevent a chronic disease or disorder. The disease or disorder could be dental caries. The disease or disorder may be a periodontal disease, or periodontal inflammation. The periodontal disease may be gingivitis. In certain embodiments, the disease or disorder may be halitosis.

In various exemplary embodiments, the disease or disorder may be tooth hypersensitivity. If the disease or disorder is tooth hypersensitivity, the composition may further comprise an additional oral care ingredient which is a desensitizing agent. The disease or disorder may be the buildup of tartar and/or calculus formation. If the disease or disorder is the buildup of tartar and/or calculus formation, the composition may further comprise an additional oral care ingredient which is a tartar control agent.

Another aspect of the present disclosure provides a method of polishing tooth enamel. The method comprises applying the oral care composition disclosed herein to the surface of the enamel. In certain embodiments, the composition may be applied in a slurry comprising the composition and a continuous liquid phase, for example wherein the liquid phase is water. In certain embodiments, the compositions disclosed herein may be applied by brushing. In various embodiments, the composition is applied by brushing for at least 2 minutes using a manual or mechanical toothbrush. In certain embodiments, the method of polishing tooth enamel is a method of whitening teeth.

In the methods for whitening teeth disclosed herein, the whitening efficacy of a composition may be measured, for example, using the CIE L*a*b* (CIELAB) scale developed by the International Commission on Illumination (CIE). CIELAB is an opponent color system based on the fact that retinal color stimuli are translated into distinctions between light and dark, red and green, and blue and yellow. CIELAB indicates these values with three axes: L*, a*, and b*. The L value indicates the lightness of a color, where L=0 is black and L=100 is white. ΔL=L_{after treatment}−Lᵢₙᵢₜᵢₐₗ. Thus, a larger positive ΔL value indicates whiter teeth. The a value ranges between +a=magenta and −a=green. The b value ranges between +b=yellow and −b=blue. The W value incorporates the L, a and b values to describe how close the measured color is to true white, where W*=(a²+b²+(L*−100)²)^{1/2}, and ΔW=W*_{treated}−W*_{baseline}. A larger negative ΔW value corresponds to greater whitening.

### Kits

A further aspect of the present disclosure provides an oral care kit comprising the oral care composition described above. The kits disclosed herein may comprise the oral care composition as disclosed herein disposed in appropriate packaging. The kits disclosed herein may optionally comprise a suitable applicator, such as a toothbrush or the like. The kits disclosed herein may also or alternatively comprise means for measuring an appropriate dosage the composition. In certain embodiments, the kits disclosed herein further comprise instructions for use of the oral care composition.

### EXAMPLE

### Example 1—Tooth Whitening Formulations Comprising Charcoal

Four toothpaste formulations were prepared, each having an abrasive silica and varying amounts of charcoal, as set forth in Table 1 below.

TABLE 1
Charcoal Formulations

| | Formulation A | Formulation B | Formulation C | Formulation D |
|---|---|---|---|---|
| | (0.5% Charcoal) | (1% Charcoal) | (3% Charcoal) | (0% Charcoal) |
| Ingredient | Wt % | Wt % | Wt % | Wt % |
| Orally acceptable carriers | QS | QS | QS | QS |
| Synthetic high-cleaning silica | 10.00 | 10.00 | 10.00 | 10.00 |
| Synthetic abrasive silica | 10.00 | 10.00 | 10.00 | 10.00 |
| Synthetic thickening silica | 5.00 | 5.00 | 5.00 | 5.00 |
| Surfactants | 5.00 | 5.00 | 5.00 | 5.00 |
| Sweeteners and Flavorants | 10.88 | 10.88 | 10.88 | 10.88 |
| Carbon/vegetable carbon | 0.50 | 1.00 | 3.00 | 0.00 |
| Preservatives | 0.50 | 0.50 | 0.50 | 0.50 |
| Thickeners | 0.65 | 0.65 | 0.65 | 0.65 |
| Total | 100.00 | 100.00 | 100.00 | 100.00 |

The formulations were then each tested for both PCR and RDA. The results are set forth below in Table 2. PCR was determined by applying clear pellicle material to a bovine tooth which was then stained with a combination of the pellicle material and tea, coffee, and FeCl₃, and subsequently treated with the composition. The change in the reflectance of the tooth surface before and after treatment was reported as the PCR value.

The RDA value was determined according to the method recommended by the American Dental Association, wherein an irradiated dentin surface was treated with a slurry of the composition to be evaluated and the level of radioactivity present in the slurry post treatment was indicative of the level of wear to the dentin surface and reported as the RDA value.

TABLE 2
PCR and RDA of Charcoal Formulations

| | Formulation A | Formulation B | Formulation C | Formulation D |
|---|---|---|---|---|
| | (0.5% | (1% | (3% | (0% |
| | Charcoal) | Charcoal) | Charcoal) | Charcoal) |
| PCR | 94 | 91 | 103 | 94 |
| RDA | 73 | 68 | 41 | 107 |

Using one-way ANOVA and SNK analyses, it was determined that there was no statistical difference (p<0.05) for the PCR of Formulations A, B, and D. Likewise, it was determined that there was no statistical difference (p<0.05) for the RDA of Formulations A and B. As shown in Table 2, increasing the amount of charcoal from 0% to 0.5% or 1% did not reduce the cleaning/whitening efficacy, as measured by PCR. Increasing the amount of charcoal to 3% increased the cleaning/whitening efficacy, as measured by PCR. Moreover, increasing the amount of charcoal from 0% to 0.5% or 1% significantly reduced the abrasivity of the composition, as measured by RDA. Likewise, increasing the amount of charcoal to 3% further reduced the abrasivity of the composition, as measured by RDA.

While the disclosure has been described with respect to specific examples including presently preferred modes of carrying out the disclosure, those skilled in the art will appreciate that there are numerous variations and permutations of the above-described systems and techniques. It is to be understood that other embodiments may be utilized and structural and functional modifications may be made without departing from the scope of the present disclosure. Thus, the scope of the disclosure should be construed broadly as set forth in the appended claims.

1. An oral care composition comprising at least one abrasive and a charcoal agent, wherein the charcoal agent is present in an amount ranging from about 0.02% to about 4% by weight based on the total weight of the oral care composition.

2. The oral care composition according to claim 1, wherein the at least one abrasive is a silica abrasive.

3. The oral care composition according to claim 2, wherein the at least one silica abrasive is chosen from high cleaning silica abrasives and standard cleaning silica abrasives.

4. The oral care composition according to claim 1, wherein the at least one abrasive is present in the oral care composition in an amount ranging from about 5% to about 25%, by weight relative to the total weight of the oral care composition.

5. The oral care composition according to claim 1, wherein the charcoal agent is present in the oral care composition in an amount ranging from about 0.5% to about 3%, by weight relative to the total weight of the oral care composition.

6. The oral care composition according to claim 1, wherein the composition has a pellicle cleaning ratio (PCR) greater than about 90.

7. The oral care composition according to claim 1, wherein the composition has a PCR greater than about 100.

8. The oral care composition according to claim 1, wherein the composition has a radioactive dentin abrasion (RDA) of less than about 200.

9. The oral care composition according to claim 1, wherein the composition has an RDA of less than about 100.

10. The oral care composition according to claim 1, wherein the composition has an RDA of less than about 50.

11. The oral care composition according to claim 1, wherein the composition has a ratio of PCR to RDA ranging from about 0.5 to about 2.5.

12. The oral care composition according to claim 1, wherein the composition has a ratio of PCR to RDA ranging from about 1.0 to about 2.5.

13. A method of making an oral care composition, the method comprising:
mixing at least one orally acceptable vehicle, at least one abrasive, and a charcoal agent to form an oral care composition,
wherein the charcoal agent is present in an amount ranging from about 0.02% to about 4% by weight based on the total weight of the oral care composition.

14. The method of claim 13, wherein the at least one orally acceptable vehicle is chosen from the group consisting of water, sorbitol, and glycerin.

15. The method of claim 13, wherein the at least one abrasive is a silica abrasive.

16. The method of claim 13, wherein the at least one abrasive is present in the oral care composition in an amount ranging from about 5% to about 25%, by weight relative to the total weight of the oral care composition.

17. The method of claim 13, wherein the charcoal agent is present in the oral care composition in an amount ranging from about 0.5% to about 3%, by weight relative to the total weight of the oral care composition.

18. The method of claim 13, wherein the composition has a PCR greater than about 90.

19. The method of claim 13, wherein the composition has an RDA of less than about 200.

20. A method of whitening and/or cleaning teeth, the method comprising:
contacting the teeth with an oral care composition comprising at least one abrasive and a charcoal agent, wherein the charcoal agent is present in an amount ranging from about 0.02% to about 4% by weight based on the total weight of the oral care composition.
